(19) United States
(12) Patent Application Publication
(10) Pub. No.: US 2026/0306149 A1
(43) Pub. Date: Oct. 8, 2026

(54) METHODS AND APPARATUS TO REMOVE TABS FROM MACHINED PARTS

(71) Applicant: Accu-Precision Inc., Littleton, CO (US)

(72) Inventors: Samuel Sebastian Camp, Boulder, CO (US); Ruby Martinez Gomez, Aurora, CO (US); Steven Dean Harris, Westminster, CO (US); David Ethan Whisnant, Orinda, CA (US); Brenton Zheng-Xian Yu, Highlands Ranch, CO (US); Ricky Carlson, Denver, CO (US); Michael W. McCue, Littleton, CO (US)

(21) Appl. No.: 19/538,069

(22) Filed: Feb. 12, 2026

Related U.S. Application Data
(60) Provisional application No. 63/783,471, filed on Apr. 4, 2025.

(51) Int. Cl. A61B 17/80; A61B 17/17

(52) U.S. Cl. CPC A61B 17/8052; A61B 17/1764

(57) ABSTRACT

Systems, apparatus, articles of manufacture, and methods to remove tabs from machined parts are disclosed. An example apparatus includes a platform including an elongate slot extending along an upper surface of the platform. The slot to receive a tab protruding from a machined part. The example apparatus further includes a rotatable cutting tool below an upper surface of the platform. The cutting tool to remove the tab from the machined part when the machined part is slid across the platform with the tab extending into the slot and beneath the upper surface of the platform.

### RELATED APPLICATION(S)

This patent claims the benefit of U.S. Provisional Patent Application No. 63/783,471, which was filed on Apr. 4, 2025. U.S. Provisional Patent Application No. 63/783,471 is hereby incorporated herein by reference in its entirety. Priority to U.S. Provisional Patent Application No. 63/783,471 is hereby claimed.

### FIELD OF THE DISCLOSURE

This disclosure relates generally to manufacturing and, more particularly, to methods and apparatus to remove tabs from machined parts.

### BACKGROUND

Many different industries rely on a variety of manufacturing processes to fabricate metal parts by removing and/or cutting away portions of a beginning piece of metal. Such manufacturing processes include computer numerical electric discharge machine (EDM) processes and computer numerical control (CNC) machining processes. To ensure parts remain in place during such processes, the parts are frequently stabilized and/or held in place during the manufacturing operations by one or more small tabs.

### BRIEF DESCRIPTION OF THE DRAWINGS

FIG. 1 illustrates a machined part that is held in place relative to a base piece of metal by one or more thin tabs.

FIG. 2 illustrates the machined part of FIG. 1 removed from the base piece of metal but with the tabs still protruding from the machined part.

FIG. 3 illustrates the machined part of FIG. 2 after removal of the tabs.

FIG. 4 illustrates an example tab removal system constructed in accordance with teaching disclosed herein.

FIG. 5 is a partially exploded view of the example tab removal system of FIG. 4.

FIG. 6 is an enlarged view of a portion of the tab removal system of FIG. 4.

FIG. 7 is a top view of the tab removal system of FIG. 4.

FIG. 8 is a front view of the example tab removal system of FIG. 4.

FIG. 9 is a left view of the tab removal system of FIG. 4.

FIG. 10 is a right view of the tab removal system of FIG. 4.

FIG. 11 illustrates an example machined part that includes an attached curved tab.

FIG. 12 is a top view of the tab removal assembly of FIGS. 4-showing an example flat tab within the example elongate slot.

FIG. 13 is a top view of the tab removal assembly of FIGS. 4-10 except that the example guide plates shown in FIG. 12 have been replaced with different example guide plates.

FIG. 14 is a top view of the tab removal assembly of FIGS. 4-10 except that one of the example guide plates shown in FIG. 12 has been replaced with another example guide plate.

FIG. 15 illustrates another example guide plate constructed in accordance with teachings disclosed herein.

FIG. 16 illustrates another example guide plate constructed in accordance with teachings disclosed herein.

FIG. 17 is a top view of the tab removal assembly of FIGS. 4-10 except that the example guide plates shown in FIG. 12 have been replaced with another example guide plate.

FIG. 18 is an exploded view of an example tab guide assembly that includes another example guide plate and an example rail attachable to the guide plate.

In general, the same reference numbers will be used throughout the drawing(s) and accompanying written description to refer to the same or like parts. The figures are not necessarily to scale.

### DETAILED DESCRIPTION

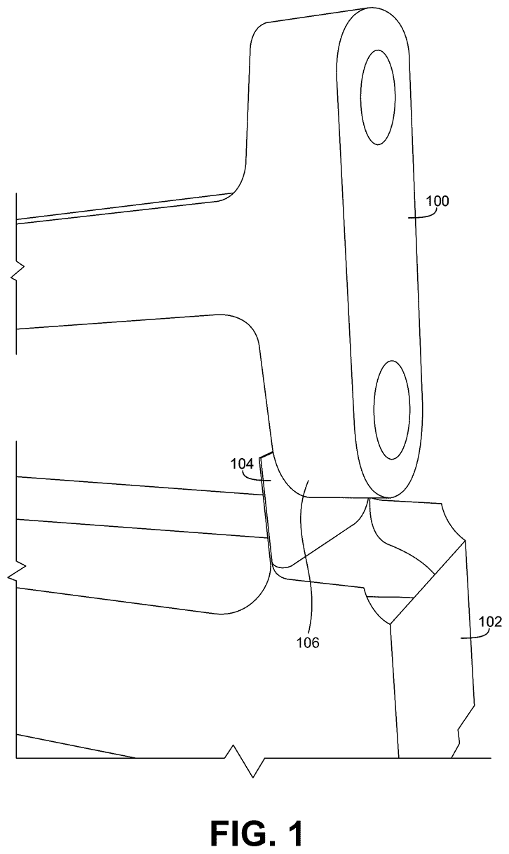

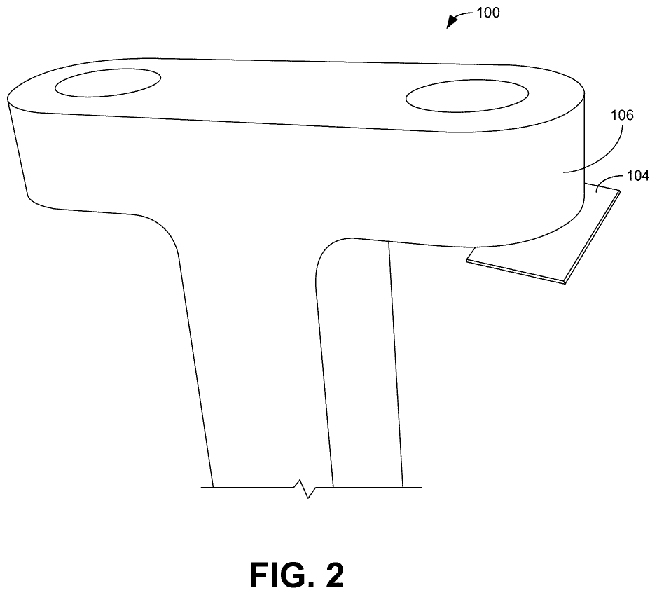

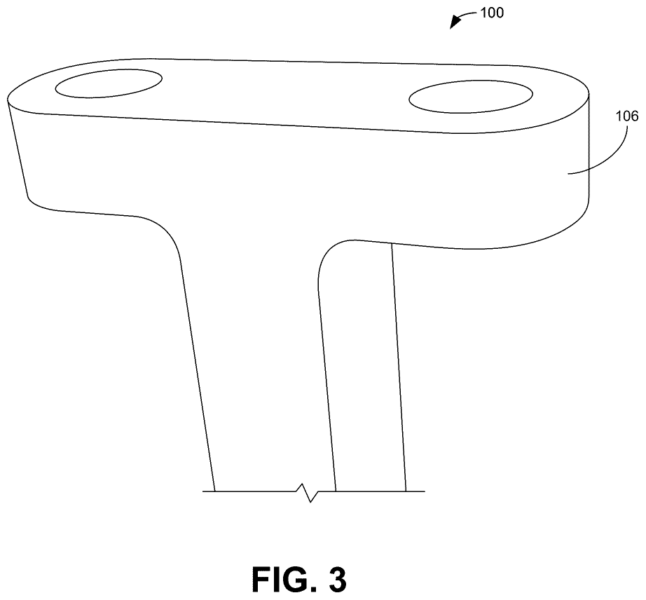

A significant challenge in manufacturing parts using metal removal technologies, such as CNC and/or wire EDM processes, is securing (e.g., holding) a part while cutting the material. Sometimes small tabs are used to stabilize the part during manufacturing operations. For instance, FIG. 1 illustrates a part 100 that is held in place relative to a base piece of metal 102 by one or more thin tabs 104 (e.g., stabilizing tabs, fixture tabs), with the base piece of metal 102 clamped into a machining tool. The part is removed from the base piece of metal 102 by cutting the tab(s) 104. Frequently, at least some portion of the tab(s) 104 is removed with the machined part 100. FIG. 2 illustrates the machined part 100 of FIG. 1 after separation from the base piece of metal 102 with a tab 104 still protruding therefrom. After separation of the part 100 from the base piece of metal 102, the tabs 104 need to be removed from the part. In many instances, the current approach to remove such tabs 104 involves manually breaking, tearing, and/or snapping the tabs 104 off by hand. Frequently, such manual removal processes can leave a small burr or other remnant of the tab 104. This is especially the case when the tab 104 protrudes away from a non-planar (e.g., curved) surface 106 of the underlying machined part 100 as shown in the illustrated example. However, slight burrs or other remnants of the tab 104 may remain even when broken off from a location corresponding to a planar surface of the machined part 100. Regardless of whether the tab 104 is removed from a planar surface or a non-planar surface, any remaining material of the tab can be subsequently removed with a hand grinder to produce the final machined part 100 as represented in FIG. 3. The resulting speed, precision, and quality of such tab removal processes can vary considerably from person to person and/or from part to part, thereby rendering this process insufficient (and potentially unsuitable) for high precision manufacturing.

Another approach to removing tabs is by repositioning and/or reclamping the part in the machining tool and using the machining tool to remove (e.g., cut and/or grind) the tabs off the part. While this can provide for much greater precision and consistency, such an approach can take significantly more time than removing the tabs 104 by hand. For instance, repositioning a machined part and setting up a tool to machine away the tabs can take as much as an hour or more because the machining tool and the part need to be set up to carefully control for all six degrees of freedom (e.g., forward/backward, up/down, left/right, pitch, roll, and yaw) and the machining tool may require programming. By contrast, manually removing tabs can take mere minutes. What is more, the additional time spent when using a machining tool to remove tabs prevents the tool from being used to machine other parts during that time. Thus, this approach is insufficient, costly, and is often not a viable option.

Examples disclosed herein enable the removal of tabs from machined parts made of high strength materials in a precise and consistent manner. Such removal can be done faster than is possible by hand. More particularly, disclosed examples are able to remove a tab with sufficient precision so as to leave less than 0.005 inches of tab material while limiting any gouging or removal of the material of the machined part to less than 0.005 inches. In some examples, the remaining material is significantly less than 0.005 inches (e.g., less than 0.002 inches, less than 0.001 inches, less than 0.001 inches, less than 0.0005 inches, etc.). This level of precision is made possible in disclosed examples through the precise positioning of a cutting tool (e.g., an end mill) adjacent to a narrow slot in a table that is just wide enough to allow the tab to extend therethrough. By inserting a tab into the slot and then sliding the tab across the cutting tool, the tab can be cleanly and quickly removed without affecting the machined part because the part is too big to fit into the slot to reach the cutting tool.

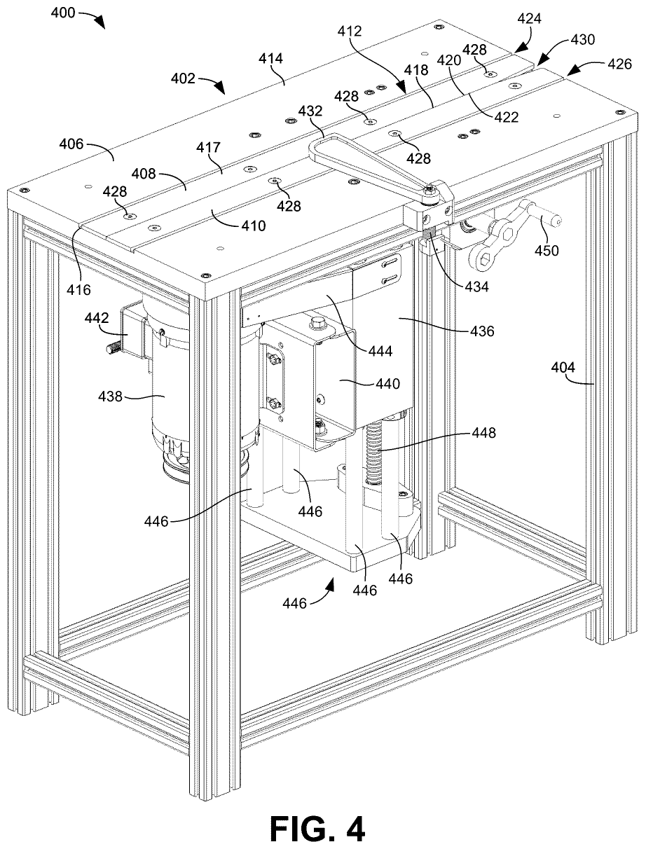

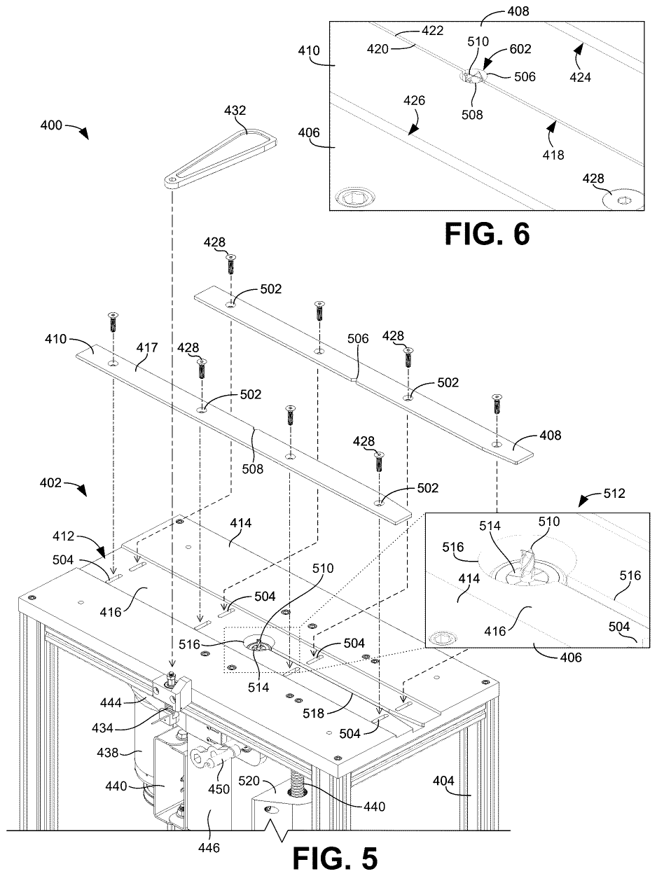

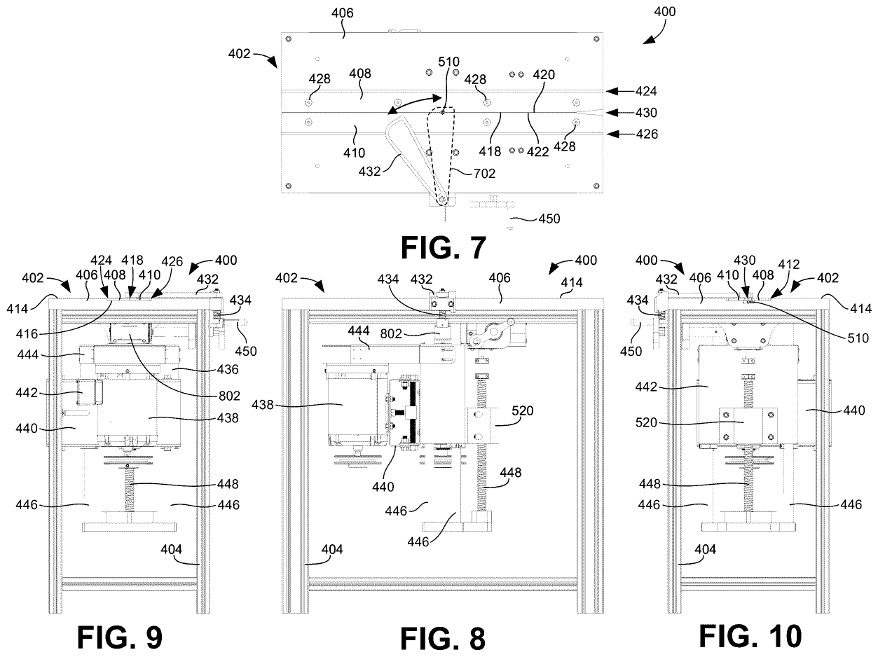

FIG. 4 illustrates an example tab removal system 400 constructed in accordance with teachings disclosed herein. FIG. 5 is a partially exploded view of the example tab removal system 400 of FIG. 4. FIG. 6 is an enlarged view of a portion of the tab removal system of FIG. 4. FIG. 7 is a top view of the tab removal system 400 of FIG. 4. FIG. 8 is a front view of the example tab removal system 400 of FIG. 4. FIG. 9 is a left view of the tab removal system 400 of FIG. 4. FIG. 10 is a right view of the tab removal system 400 of FIG. 4. In this example, the tab removal system 400 includes an upper platform 402 supported by a frame 404. In this example, the frame 404 includes four legs, although other numbers of legs are contemplated. In some examples, one or more sides of the frame (e.g., the gaps between the legs) are covered by panels to protect operators from the equipment located underneath the upper platform 402. Additionally or alternatively, in some examples, the legs and/or associated panels can be replaced by a box, housing, and/or other frame structure on which the upper platform 402 is attached. In some examples, the frame 404 is tall enough for the tab removal system 400 to operate as a free-standing table that rests on the floor. In other examples, the frame 404 is shortened so that the system 400 is to rest on an underlying table, counter, and/or any other suitable surface.

In the illustrated example, the upper platform 402 includes a base 406 and two guide plates (e.g., a first guide plate 408 and a second guide plate 410). In some examples, both the base 406 and the guide plates 408, 410 are made of steel (e.g., air-hardened A2 tool steel) or other suitable material. In this example, the two guide plates 408, 410 are mirror images of one other. In other examples, the first guide plate 408 has a size and/or shape that is different from the second guide plate 410. As shown in the illustrated examples, the base 406 defines a bulk (e.g., a majority) of the platform 402 with the guide plates 408, 410 positioned within a recessed region 412 in the base 406. That is, the base 406 includes a first surface 414 (e.g., a top surface, an outer surface, an uppermost surface) and a second surface 416 (e.g., a recessed surface) that is recessed relative to the first surface 414. As shown in FIG. 5, the second surface 416 defines the recessed region 412. In this example, the recessed region 412 is a rectangular trench that extends an entire length of the base 406. In other examples, the recessed region 412 extends less than the full length of the base 406. Additionally or alternatively, in some examples, the recessed region 412 is a different shape than what is shown (e.g., round, curved, etc.). In some examples, the recessed region 412 is omitted and the guide plates 408, 410 are attached to the first surface 414 (e.g., the top surface) of the base 406.

In some examples, the depth of the recessed region 412 (e.g., the distance the second surface 416 is recessed or inset relative to the first surface 414) is approximately equal to a thickness of the guide plates 408, 410. As a result, in some examples, a top surface 417 of the guide plates 408, 410 is substantially flush with (e.g., within 0.1 inches of) the first surface 414 of the base 406. In other examples, the thickness of the guide plates 408, 410 can be greater or less than the depth of the recessed region 412.

In some examples, the width of the recessed region 412 is greater than a combined width of both the first and second guide plates 408, 410 so that the guide plates 408, 410 can be positioned within the recessed region 412 and spaced apart with a gap therebetween defining an elongate slot 418 (e.g., a first elongate slot) extending along a length of the platform 402. That is, in some examples, a first edge 420 of the first guide plate 408 defines a first side of the elongate slot 418 and a second edge 422 of the second guide plate 410 defines a second side of the elongate slot 418 opposite the first side. Further, in some examples, the recessed region 412 is dimensioned wide enough to define additional gaps 424, 426 along the edges of the guide plates 408, 410 facing away from the elongate slot 418 (e.g., towards sidewalls of the recessed region 412).

In some examples, the guide plates 408, 410 are secured (e.g., attached, connected) to the base 406 using one or more threaded fasteners 428 (e.g., bolts, screws, etc.) to enable the selective detachment, connection, and/or repositioning of the guide plates 408, 410 relative to the base 406. As shown in the exploded view of FIG. 5, the threaded fasteners 428 extend through first holes 502 in the guide plates 408, 410 and second holes 504 in the base 406. In this example, the second holes 504 are elongate in a direction transverse to the length of the recessed region 412 and the length of the guide plates 408, 410. The orientation and elongate shape of the second holes 504 enable the placement of the threaded fasteners 428 to be adjusted along the length of the elongate second holes 504. As a result, the position of the guide plates 408, 410 can be adjusted relative to one another and relative to the base 406. That is, the elongate second holes 504 enable the guide plates 408, 410 to be selectively shiftable so as to adjust the width of the elongate slot 418 defined between the guide plates 408, 410. Such adjustment correspondingly adjusts the widths of the gaps 424, 426 along the outward facing edges of the guide plates 408, 410. Thus, in the illustrated example, the maximum width of the elongate slot 418 corresponds to when the guide plates 408, 410 are shifted outwards to abut the sidewalls of the recessed region 412. In some examples, the first holes 502 can be elongate in addition to or instead of the second holes 504 to enable shifting of the guide plates 408, 410 relative to one another and/or relative to the base 406.

In some examples, an operator may shift the guide plates 408, 410 along the elongate second holes 504 to adjust the width of the elongate slot 418 to be slightly larger than a thickness of a tab to be removed from a machined part (e.g., the tab 104 shown in FIG. 2). In this manner, an operator can then insert the tab 104 into the elongate slot 418 and slide the tab 104 along a length of the slot 418. That is, the guide plates 408, 410 serve to guide a tab 104 along a length of the platform 42. In some examples, the guide plates 408, 410 include a tapered end to define an enlarged opening 430 (e.g., a V-shaped opening) at an end of the elongate slot 418 to facilitate insertion of the tab 104 into the slot 418. In some examples, a similar enlarged opening 430 is at both ends of the elongate slot 418. In some examples, the enlarged opening 430 can be a different size and/or different shape from what is shown in the illustrated example. In some examples, the enlarged opening 430 is omitted.

In some examples, as shown in FIGS. 5 and 6, the inner facing edges 420, 422 of the guide plates 408, 410 (defining the elongate slot 418) include corresponding notches 506, 508 at a location spaced apart from the ends of the guide plates 408, 410 (e.g., near a middle of the guide plates 408, 410). In this example, as shown in the illustrated example of FIG. 6, the notches 506, 508 are aligned to define different portions of an opening 602 in the platform 402 within which a rotatable cutting tool 510 is positioned. An enlarged view of the cutting tool 510 (with the guide plates 408, 410 removed) is shown in the inset image 512 of FIG. 5 (which corresponds to the same portion of the tab removal system 400 shown in FIG. 6). In this example, the cutting tool 510 is an end mill. However, any other rotatable tool can be employed. In this example, the cutting tool 510 is 0.375 inches in diameter. However, in other examples, the cutting tool 510 can be larger or smaller.

In some examples, the uppermost tip of the cutting tool is to be flush with or below the top surfaces 417 of the guide plates 408, 410. In this arrangement, when a tab of a machined part (e.g., the tab 104 of the machined part 100 of FIG. 2) is inserted and slid along the elongate slot 418, the tab 104 will be removed (e.g., cut away) by the rotating cutting tool 510. That is, the slot 418 (defined by the guide plates 408, 410) guides the tab 104 along a path into contact with the cutting tool 510. Due to the narrow width of the slot 418 and the relatively small size of the opening 602 (only slightly larger than the size of the cutting tool 510), the machined part 100 is prevented from extending below the top surface 417 of the guide plates 408, 410. As a result, the cutting tool 510 can remove the tab 104 without affecting (e.g., gouging) the machined part 100 in a precise and consistent manner. Setting the height of the cutting tool 510 relative to the work surface of the platform 402 (e.g., the top surface 417 of the guide plates 408, 410) and positioning the guide plates 408, 410 to define the elongate slot 418 that guides the path of a tab 104 to be removed automatically accounts for most of the six degrees of freedom that would need to be carefully controlled if the tab 104 were to be removed by a machining tool. That is, so long as an operator keeps the machined part 100 flush against the work surface with the tab 104 in the slot 418, the tab 104 will be removed as the part 100 is slid across the work surface toward the cutting tool 510. This is especially simple when the tab protrudes from a planar surface because the planar surface can rest directly on the work surface of the platform 402. In cases where the tab protrudes from a non-planar surface (e.g., the curved surface 106 shown in FIGS. 1-3), the operator may need to rotate the machined part 100 as the tab 104 is removed by the cutting tool 510. Regardless of the particular shape of the machined part 100 or the location of the tab 104 (e.g., whether protruding from a planar surface or a non-planar surface), there is little to no set-up time to use the example tab removal system 400 and it can be used on a large variety of parts without having to make any adjustments, thereby providing a much faster and more precise approach to remove tabs than has been possible in the past.

In some examples, the tab removal system 400 includes a guard arm 432 that is to be positioned above the platform 402 and rotatable relative to the platform 402. More particularly, in some examples, the guard arm 432 is rotatable between a first position (shown in FIG. 4 and shown by dashed lines 702 in FIG. 7) and a second position (shown in solid lines in FIG. 7). As shown in the illustrated example, the guard arm 432 is to extend overtop of the cutting tool 510 when in the first position and to be spaced apart from the cutting tool 510 in the second position. In some examples, a spring 434 is operatively coupled to the guard arm 432 to urge the guard arm 432 towards the first position while permitting the guard arm 432 to be pushed to the second position in response to a machined part (e.g., the part 100 of FIG. 2) being slid across the platform 402 and into contact with the guard arm 432.

As shown in the inset image 512 of FIG. 5, the cutting tool 510 is affixed in a collet 514 positioned within and/or aligned with an opening 516 in the second surface 416 of the base 406. In this example, the opening 516 is aligned with and, thus, defines an extension of the opening 602 in the platform 402 defined by the notches 506, 508 in the guide plates 408, 410. In some examples, the base 406 includes a second elongate slot 518 in the second surface 416 of the base 406. As shown in FIG. 5, the second elongate slot 518 is generally aligned with the first elongate slot 418 defined by the inner facing edges 420, 422 of the guide plates 408, 410. In this manner, tabs 104 that protrude from a machined part 100 by an extent greater than a thickness of the guide plates 408, 410 can extend into the second elongate slot 418 as the guide plates 408, 410 guide the tabs 104 along the first elongate slot 418 toward the cutting tool 510. In some examples, the second elongate slot 518 is omitted and tabs 104 are limited to extending into the first elongate slot 418 a distance corresponding to a thickness of the guide plates 408, 410 (which may be greater than what is shown in the illustrated example).

In this example, the cutting tool 510 and the associated collet 514 are spun or rotated about a spindle 802 (shown in FIGS. 8 and 9) that is substantially perpendicular to the first and/or second surfaces 414, 416 of the base 406. As such, inasmuch as the cutting tool 510 is positioned at or below the top surfaces 417 of the guide plates 408, 410 (e.g., an upper surface of the platform 402), the cutting tool 510 rotates in a plane parallel to and below the upper surface of the platform 402. As used herein, substantially perpendicular means within 5 degrees of exactly perpendicular. In some examples, the spindle 802 is supported within a spindle housing 436 that is attached to an associated motor 438 via an intervening bracket assembly 440. In some examples, the bracket assembly 440 enables adjustment of the position of the motor 438 relative to the spindle housing 436. In this example, the motor 438 is operatively coupled (e.g., via a belt) to the spindle 802 to drive rotation of the associated cutting tool 510 and collet 514. In some examples, the motor 438 includes and/or is associated with a control box 442 by which an operator can adjust and/or control the speed at which the cutting tool 510 spins for use in connection with different sizes, shapes, thicknesses, and/or materials (e.g., high strength materials) of the tabs 104 to be removed from corresponding machined parts 100. In some examples, a splash guard 444 is provided to cover and/or protect the motor 438.

In some examples, the tab removal system 400 includes a lift assembly 446 that enables an operator to raise or lower the cutting tool 510 relative to the platform 402 by raising or lowering the spindle housing 436 and the motor 438. More particularly, as shown in the illustrated example, the spindle housing 436 is positioned to glide along one or more guide shafts 448 of the lift assembly 446. Further, the spindle housing 436 is attached to a lift carriage 520 (shown in FIGS. 5, 8, and 10) of the lift assembly 446 that is operatively coupled to a threaded shaft 448. In some examples, the threaded shaft 448 is manually rotated using an operatively coupled crank 450. Depending on the direction of rotation of the shaft 448, the lift carriage 520 will either raise or lower along the threads of the shaft 448, thereby causing the spindle housing 436, the motor 438, and the associating cutting tool 510 to raise or lower. In this manner, the position of the cutting tool 510 can be precisely located at any suitable height relative to the upper platform 402 (e.g., coincident with the top surface 417 of the guide plates 408, 410).

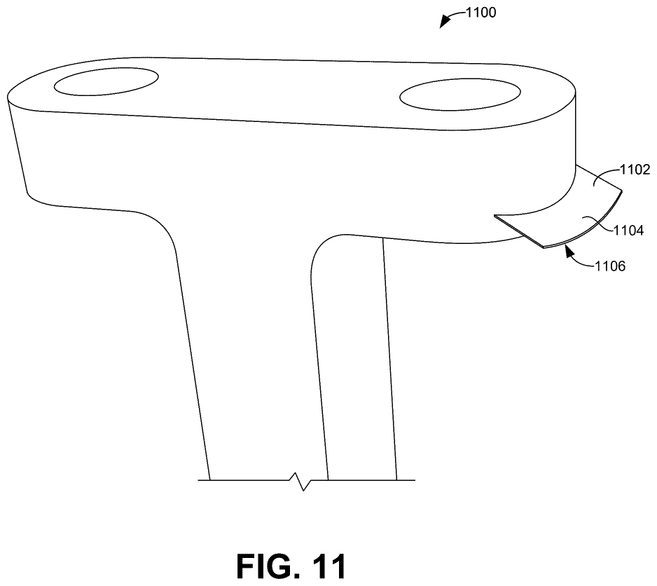

As discussed above, in some examples, the distance between the inner facing edges 420, 422 of the guide plates 408, 410 may be adjusted by shifting the threaded fasteners 428 along the length of the elongate second holes 504 in the recessed region 412 of the base 406 to accommodate tabs 104 of different thicknesses. In some examples, the spacing between the guide plates 408, 410 can accommodate tabs that are curved rather than flat. Specifically, FIG. 11 illustrates an example machined part 1100 that includes a curved tab 1102 still attached. As shown in FIG. 11, the curved tab includes a concave surface 1104 and a convex surface 1106 opposite the concave surface 1104. In some examples, the radius of curvature of the curved tab 1102 can be any suitable radius. Such curved tabs 1102 are useful to stabilize machined parts during machining operations because the curved nature of the tab 1102 provides added strength relative to a flat tab, such as the tab 104 shown in FIG. 2. Additionally or alternatively, the curved tab 1102 can be fabricated thinner than a flat tab 104 while still providing adequate support for the machined part 1100. The possibility of the curved tab 1102 being thinner than a flat tab 104 can make it easier to remove the curved tab 1102 because there is less material to remove.

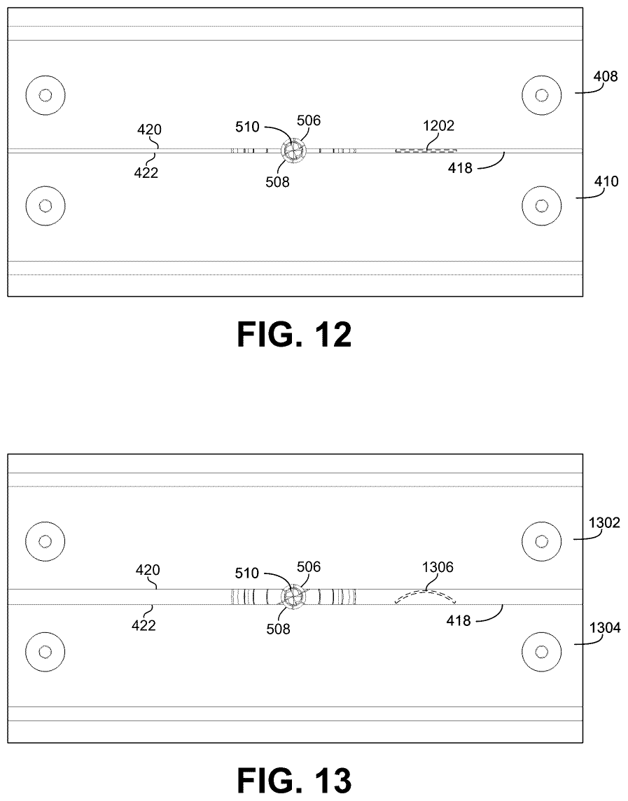

While the shape of the curved tab 1102 of FIG. 11 can provide added strength, the shape of the curved tab 1102 prevents the tab from fitting in the elongate slot 418 of the tab removal system 400 of FIGS. 4-10 when the slot 418 is adjusted with a width only slightly larger than a flat tab. One option to resolve this issue is to widen the elongate slot 418 by shifting the guide plates 408, 410 apart until the curved tab 1102 is able to fit through the slot 418. However, this would also move the notches 506, 508 adjacent to the cutting tool 510 farther apart, thereby giving rise to the possibility of the machined part 100 extending into the associated opening 602 and inadvertently gets cut or gouged by the cutting tool 510. Accordingly, in some examples, different guide plates may be used that have a smaller width and/or a smaller notch on the inner facing edges of the associated guide plates. More particularly, FIG. 12 is a top view of the elongate slot 418 of the tab removal system 400 of FIGS. 4-10 with an example flat tab 1202 represented by dashed lines. As shown, the width of the slot 418 is slightly larger than the flat tab 1202 such that a curved tab would not fit. By contrast, FIG. 13 is a top view of the tab removal system 400 of FIGS. 4-10 except that the guide plates shown in FIG. 12 have been replaced with different guide plates 1302, 1304 (e.g., third and fourth guide plates). As shown, the distance between the inner facing edges 420, 422 of the third and fourth guide plates 1302, 1304 (defining the width of the elongate slot 418) in FIG. 13 is greater than the distance between the inner facing edges 420, 422 of the guide plates 408, 410 shown in FIG. 12. The greater distance in the example of FIG. 13 enables a curved tab 1306 (represented by dashed line) to fit within and slide along the elongate slot 418 towards the cutting tool 510 to be removed. Significantly, although the third and fourth guide plates 1302, 1304 are spaced farther apart in FIG. 13, the notches 506, 508 remain the same distance from the cutting tool 510 because the notches 506, 508 in FIG. 13 are smaller than the notches 506, 508 in FIG. 12. Thus, in some examples, rather than adjusting the position of the guide plates 408, 410 used in the example tab removal system 400 of FIGS. 4-10 and 12 for different sized and/or shaped tabs, different shaped guide plates may be swapped out and/or replaced for the guide plates 408, 410 that are more suitably dimensioned for a given size and/or shape of tab.

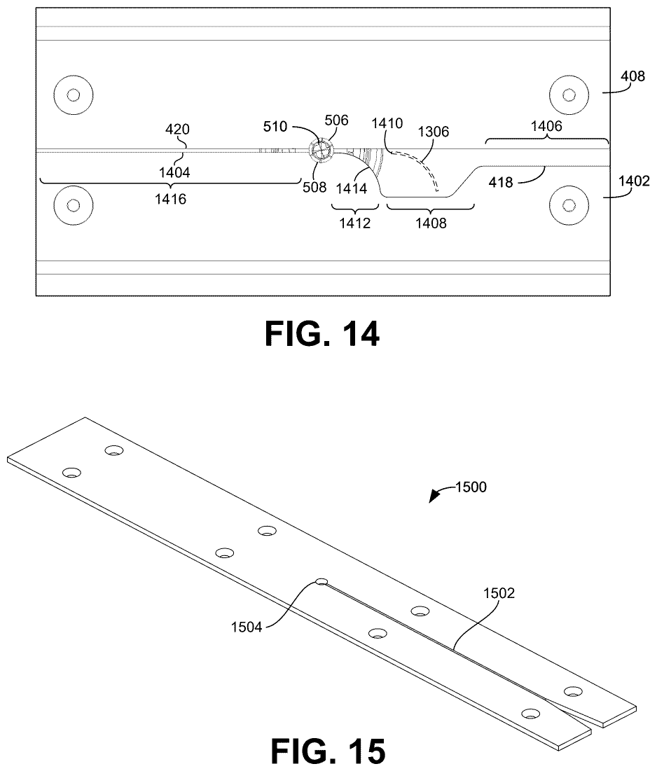

FIG. 14 illustrates the first guide plate 408 used in combination with an example fifth guide plate 1402 that has replaced the second guide plate 410 (shown in FIG. 12). The example fifth guide plate 1402 facilitates the removal of a curved tab (e.g., the curved tab 1306 shown in FIG. 13) without the need for spacing the guide plates 408, 1402 at the location of the cutting tool 510 as far apart as shown in FIG. 13. Specifically, in this example, the fifth guide plate 1402 includes a third edge 1404 that faces towards the first edge 420 of the first guide plate 408 to define the elongate slot 418. However, as shown in the illustrated examples, different portions of the third edge 1404 are non-linear and/or spaced different distances from the first edge 420. More particularly, along a first portion 1406, the distance between the facing edges 420, 1404 is similar to what is shown in FIG. 13 to enable the curved tab 1306 to slide along the length of the elongate slot 418. The shape of the third edge 1404 along a second portion 1408 is spaced farther away from the first edge 420 to define a space in which the curved tab can be rotated so that a leading edge 1410 of the tab 1306 is aligned with (e.g., facing in a direction substantially parallel to) the first edge 420 of the first guide plate 408. A third portion 1412 of the third edge 1404 includes a curve 1414 that corresponds to (e.g., matches, is complementary to, has a similar radius of curvature to) the curved tab 1306. Finally, a fourth portion 1416 on the opposite side of the cutting tool 510 to the other portions defines a distance between the facing edges 420, 1404 similar to what is shown in FIG. 12. In the illustrated example of FIG. 14, the curve 1414 defines a lead-in surface that the curved tab 1306 can be slid along as the tab 1306 approaches the cutting tool 510 for precise control and to narrow the gap between the edges 420, 1404 at the location of the cutting tool 510 as compared to the example shown in FIG. 13. Narrowing the gap between the edges 420, 1404 in this manner can reduce the likelihood of a machine part (e.g., the machined part 100) being gouged by the cutting tool 510.

In some examples, the first portion 1406 of the elongate slot 418 shown in FIG. 14 has the same width as the second portion 1408. That is, in some examples, the slot 418 is relatively wide nearly all of its length from an outer end to the cutting tool 510 except for the curved surface 1414 in the third portion 1412 that narrows the slot to guide a curved tab into engagement with the cutting tool 510. Notably, although FIGS. 13 and 14 are described as being used to remove a curved tab, straight tabs can also be removed using such arrangements of the tab removal system 400.

Many other variations to the guide plates 408, 410, 1302, 1304, 1402 disclosed herein are possible. For instance, inasmuch as the tab 104 is removed once the machined part 100 is slid across the platform up to the cutting tool 510, in some examples, the elongate slot 418 also ends at the cutting tool 510. That is, in some examples, the first and fifth guide plates 408, 1402 in FIG. 14 are modified so that the facing edges 420, 1404 along the fourth portion 1416 are directly abutting. Similar adjustments can be made in the illustrated examples in FIGS. 12 and 13. Alternatively, in some examples, the pair of guide plates in any one of FIGS. 12-14 (e.g., the first and second guide plates 408, 410 in FIG. 12, the third and fourth guide plates 1302, 1304 in FIG. 13, the first and fifth guide plates in FIG. 14) can be replaced with a single plate having a combined width of both guide plates 408, 410, 1302, 1304, 1402 of the illustrated examples and a slit extending from one end to the middle of the plate to guide the tab 104 on the machined part 100 towards the cutting tool 510. This is represented in the illustrated example of FIG. 15, which shows a single guide plate 1500 that can replace and provide the same functionality as the first and second guide plates 408, 410 shown in FIG. 12. As shown in the illustrated example, the single guide plate 1500 includes an elongate slot 1502 similar in shape to the elongate slot 418 shown in FIGS. 6, 7, and 12, except that the slot 1502 is closed at one end (e.g., adjacent to the cutting tool 510) with the other end extending to the outer edge (e.g., perimeter) of the guide plate 1500. More particularly, in this example, the elongate slot 1502 is closed or ends at an opening 1504 (similar to the opening 602 in FIG. 6) that is to surround the cutting tool 510. In other examples, the elongate slot 1502 in FIG. 15 may extend beyond the opening 1504 but still end before reaching the opposite end of the guide plate 1500. The elongate slot 1502 can be modified (e.g., customized) to any suitable shape (e.g., to provide the structure and functionality of the third and fourth guide plates 1302, 1304 shown in FIG. 13 or the first and fifth guide plates 408, 1402 shown in FIG. 14).

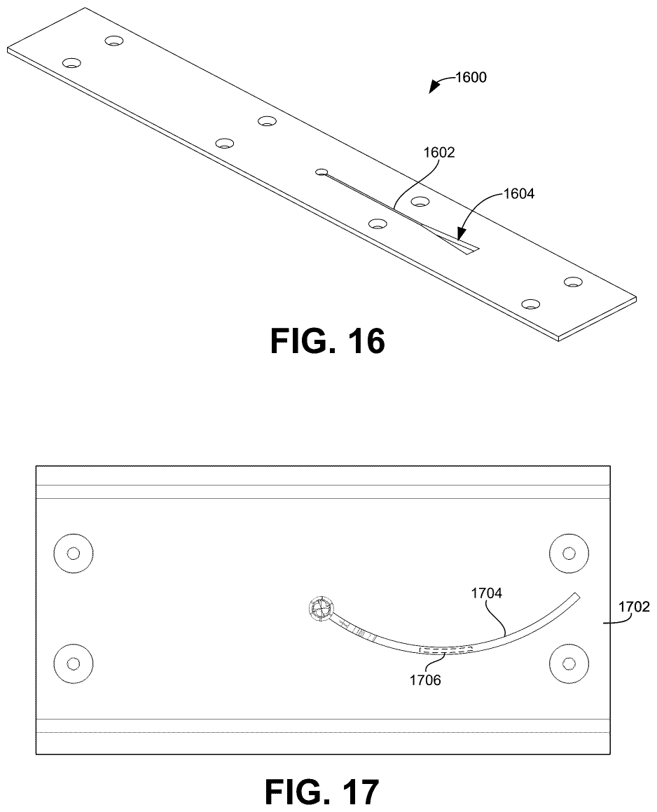

FIG. 16 illustrates another example guide plate 1600 that includes an example elongate slot 1602 that is closed at both ends. That is, unlike what is shown in the previous illustrated examples, the elongate slot 1602 of FIG. 16 does not extend to the end of the platform 402 but begins at a point closer to the cutting tool 510. In some such examples, the beginning of the elongate slot 418 (e.g., an end of the slot distal from the cutting tool 510) is defined by an enlarged opening 1604 to facilitate insertion of a tab (e.g., the tab 104) into the slot 1602). In other examples, the enlarged opening 1604 is omitted. In some examples, the elongate slot 1602 can be modified (e.g., customized) to any suitable shape to guide any type of tab along the slot 1602 towards the cutting tool 510 as detailed above. For instance, FIG. 17 illustrates another example guide plate 1702 that has replaced the other guide plates 408, 410, 1302, 1304, 1402 discussed above in connection with FIGS. 12-14. In this example, similar to the example shown in FIG. 16, the example guide plate 1702 of FIG. 17 includes an elongate slot 1704 that is closed at both ends. However, unlike what is shown in FIG. 16, the elongate slot 1704 of FIG. 17 is non-linear (e.g., curved) to match a corresponding shape of a tab (e.g., a curved tab 1706).

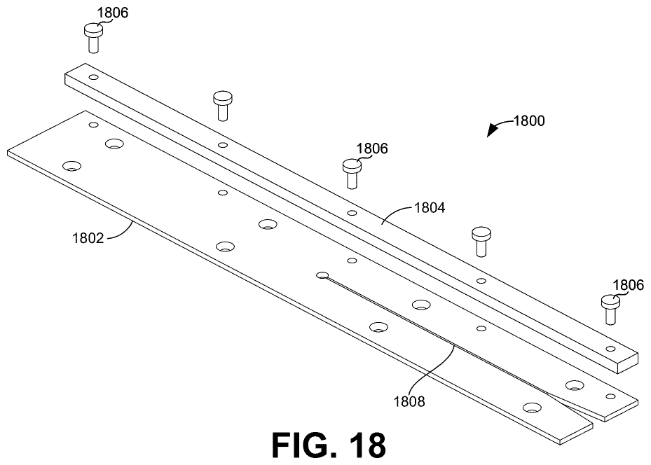

FIG. 18 is an exploded view of an example tab guide assembly 1800 that includes another example guide plate 1802 and an example rail 1804 attachable to the guide plate 1802 using one or more fasteners 1806. In this example, the guide plate 1802 is substantially the same as the guide plate 1500 of FIG. 15 except that the guide plate 1802 of FIG. 18 includes additional holes to receive the fasteners 1806. Any of the other example guide plates 408, 410, 1302, 1304, 1402, 1600, 1702 can be modified to enable attachment of a rail. In the example of FIG. 18, the rail 1804 attaches to the guide plate 1802 so as to protrude upwards from the guide plate 1802 to define a ridge or lip that a machine part can be laterally pressed against as the machine part is pushed along the length of the guide plate 1802 with a tab to be removed travelling within the elongate slot 1808. In some examples, the rail 1804 extends less than the full length of the guide plate 1802. In some examples, either the holes in the rail 1804 or the corresponding holes in the guide plate 1802 are elongated in a direction transverse to a length of the guide plate 1802. In this manner, the rail 1804 can be laterally adjusted to different distances from the elongate slot 1808. In other examples, the rail is permanently affixed to the guide plate 1802. In some examples, the rail 1804 is an integral part of the guide plate 1802. In some examples, two separate rails extend from the guide plate 1802 with one on either side of the elongate slot 1808.

Although many different example guide plates 408, 410, 1302, 1304, 1402, 1600, 1702, 1802 have been described in detail above, this disclosure is not limited thereto but covers any suitable variation in the size, shape, and/or design of a guide plate and/or the resulting elongate slot used to guide a tab towards the cutting tool 510 in the example tab removal system 400, While many guide plates may be capable of removing many different tabs from many different machined parts, some tab on some parts may present unique circumstances that need guide plates with a particular design to facilitate the removal of such tabs. Examples disclosed herein enable the quick and simple replacement of one or more guide plates with others on the underlying base 406 of the platform 402 to address such unique circumstances. Furthermore, if a unique tab and/or associated machined part does not fit any existing guide plates an operator has on hand, it is relatively easy to have new guide plates created (e.g., customized) to facilitate the removal of the unique tabs without needing an entirely new tab removal system 400.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc., may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, or (7) A with B and with C. As used herein in the context of describing structures, components, items, objects and/or things, the phrase “at least one of A and B” is intended to refer to implementations including any of (1) at least one A, (2) at least one B, or (3) at least one A and at least one B. Similarly, as used herein in the context of describing structures, components, items, objects and/or things, the phrase “at least one of A or B” is intended to refer to implementations including any of (1) at least one A, (2) at least one B, or (3) at least one A and at least one B. As used herein in the context of describing the performance or execution of processes, instructions, actions, activities, etc., the phrase “at least one of A and B” is intended to refer to implementations including any of (1) at least one A, (2) at least one B, or (3) at least one A and at least one B. Similarly, as used herein in the context of describing the performance or execution of processes, instructions, actions, activities, etc., the phrase “at least one of A or B” is intended to refer to implementations including any of (1) at least one A, (2) at least one B, or (3) at least one A and at least one B.

As used herein, singular references (e.g., “a”, “an”, “first”, “second”, etc.) do not exclude a plurality. The term “a” or “an” object, as used herein, refers to one or more of that object. The terms “a” (or “an”), “one or more”, and “at least one” are used interchangeably herein. Furthermore, although individually listed, a plurality of means, elements, or actions may be implemented by, e.g., the same entity or object. Additionally, although individual features may be included in different examples or claims, these may possibly be combined, and the inclusion in different examples or claims does not imply that a combination of features is not feasible and/or advantageous.

As used herein, unless otherwise stated, the term “above” describes the relationship of two parts relative to Earth. A first part is above a second part, if the second part has at least one part between Earth and the first part. Likewise, as used herein, a first part is “below” a second part when the first part is closer to the Earth than the second part. As noted above, a first part can be above or below a second part with one or more of: other parts therebetween, without other parts therebetween, with the first and second parts touching, or without the first and second parts being in direct contact with one another.

As used in this patent, stating that any part (e.g., a layer, film, area, region, or plate) is in any way on (e.g., positioned on, located on, disposed on, or formed on, etc.) another part, indicates that the referenced part is either in contact with the other part, or that the referenced part is above the other part with one or more intermediate part(s) located therebetween.

As used herein, connection references (e.g., attached, coupled, connected, and joined) may include intermediate members between the elements referenced by the connection reference and/or relative movement between those elements unless otherwise indicated. As such, connection references do not necessarily infer that two elements are directly connected and/or in fixed relation to each other. As used herein, stating that any part is in “contact” with another part is defined to mean that there is no intermediate part between the two parts.

Unless specifically stated otherwise, descriptors such as “first,” “second,” “third,” etc., are used herein without imputing or otherwise indicating any meaning of priority, physical order, arrangement in a list, and/or ordering in any way, but are merely used as labels and/or arbitrary names to distinguish elements for ease of understanding the disclosed examples. In some examples, the descriptor “first” may be used to refer to an element in the detailed description, while the same element may be referred to in a claim with a different descriptor such as “second” or “third.” In such instances, it should be understood that such descriptors are used merely for identifying those elements distinctly within the context of the discussion (e.g., within a claim) in which the elements might, for example, otherwise share a same name.

As used herein, “approximately” and “about” modify their subjects/values to recognize the potential presence of variations that occur in real world applications. For example, “approximately” and “about” may modify dimensions that may not be exact due to manufacturing tolerances and/or other real world imperfections as will be understood by persons of ordinary skill in the art. For example, “approximately” and “about” may indicate such dimensions may be within a tolerance range of +/−10% unless otherwise specified herein.

From the foregoing, it will be appreciated that example systems, apparatus, articles of manufacture, and methods have been disclosed that enable the removal of tabs from machined parts in a quick, precise, and consistent manner. Specifically, disclosed examples involve a platform having an elongate slot into which a tab is inserted while still attached to a machined part. The machined part is slid along the surface of the platform with the tab being guided within the elongate slot toward a spinning cutting tool within an opening in the platform. The cutting tool is positioned to be coincident with or below the surface of the platform so as to cut away the tab without affecting the machined part.

Further examples and combinations thereof include the following:

Example 1 includes an apparatus comprising a platform including an elongate slot extending along an upper surface of the platform, the slot to receive a tab protruding from a machined part, and a rotatable cutting tool below an upper surface of the platform, the cutting tool to remove the tab from the machined part when the machined part is slid across the platform with the tab extending into the slot and beneath the upper surface of the platform.

Example 2 includes any preceding clause(s) of example 1, wherein the platform includes a base and a guide plate selectively connectable to and detachable from the base, the guide plate to be secured to the base, the guide plate to define a portion of the upper surface of the platform along which the machined part is to slide, an edge of the guide plate to define an edge of the slot.

Example 3 includes any preceding clause(s) of any one or more of examples 1-2, wherein the guide plate is a first guide plate, the apparatus including a second guide plate selectively connectable to and detachable from the base, the first and second guide plates to be secured to the base with a gap between the first and second guide plates, a width of the gap defining a width of the slot.

Example 4 includes any preceding clause(s) of any one or more of examples 1-3, wherein the first guide plate has a first shape and the second guide plate has a second shape, the second shape mirroring the first shape.

Example 5 includes any preceding clause(s) of any one or more of examples 1-4, including a third guide plate to replace at least one of the first guide plate or the second guide plate, the third guide plate having a third shape that is different from the first shape and different from a second shape.

Example 6 includes any preceding clause(s) of any one or more of examples 1-5, wherein at least one of the first guide plate or the second guide plate is selectively shiftable relative to the base in a direction transverse to a length of the slot to adjust the width of the slot.

Example 7 includes any preceding clause(s) of any one or more of examples 1-6, wherein the base includes a first surface and a second surface that is recessed relative to the first surface, the second surface defining a recessed region of the base, the guide plate to be within the recessed region.

Example 8 includes any preceding clause(s) of any one or more of examples 1-7, wherein a top surface of the guide plate is to be substantially flush with the first surface of the base when secured within the recessed region.

Example 9 includes any preceding clause(s) of any one or more of examples 1-8, wherein the slot extends to an end of the platform.

Example 10 includes any preceding clause(s) of any one or more of examples 1-9, wherein the slot includes an enlarged opening at a first end of the slot to facilitate insertion of the tab into the slot.

Example 11 includes any preceding clause(s) of any one or more of examples 1-10, wherein an uppermost tip of the cutting tool is to be flush with or below the upper surface of the platform.

Example 12 includes any preceding clause(s) of any one or more of examples 1-11, wherein a height of the cutting tool is adjustable relative to the upper surface of the platform.

Example 13 includes any preceding clause(s) of any one or more of examples 1-12, including a motor to cause the cutting tool to rotate on a spindle, the spindle substantially perpendicular to the upper surface of the platform.

Example 14 includes any preceding clause(s) of any one or more of examples 1-13, including a guard arm, the guard arm rotatable relative to the platform between a first position and a second position, the guard arm to extend over the cutting tool in the first position, the guard arm to be spaced apart from the cutting tool in the second position.

Example 15 includes any preceding clause(s) of any one or more of examples 1-14, including a spring to urge the guard arm toward the first position while permitting the guard arm to move to the second position in response to the machined part being slid across the platform.

Example 16 includes any preceding clause(s) of any one or more of examples 1-15, wherein the tab is a curved tab, and the slot has a shape to receive the curved tab.

Example 17 includes an apparatus comprising a first plate including a first edge, a second plate including a second edge, a base to support the first and second plates with the first edge facing towards the second edge, a distance between the first and second edges defining a slot to guide a tab protruding from a machined part, and a cutting tool to be located along the slot so as to remove the tab from the machined part as the tab is guided along the slot.

Example 18 includes any preceding clause(s) of example 17, wherein the first plate includes a first notch along the first edge, and the second plate includes a second notch along the second edge, the first notch to align with the second notch to define an opening therebetween, the cutting tool in the opening.

Example 19 includes an apparatus comprising a cutting tool, a motor to cause the cutting tool to spin, and a platform including an elongate slot to guide a tab protruding from a machined part towards the cutting tool, an uppermost tip of the cutting tool to be at or below an upper surface of the platform.

Example 20 includes any preceding clause(s) of example 19, including a first guide plate to define a first side of the elongate slot, and a second guide plate to define a second side of the elongate slot, the cutting tool to be between the first and second guide plates.

The following claims are hereby incorporated into this Detailed Description by this reference. Although certain example systems, apparatus, articles of manufacture, and methods have been disclosed herein, the scope of coverage of this patent is not limited thereto. On the contrary, this patent covers all systems, apparatus, articles of manufacture, and methods fairly falling within the scope of the claims of this patent.

What is claimed is:

1. An apparatus comprising:
a platform including an elongate slot extending along an upper surface of the platform, the slot to receive a tab protruding from a machined part; and
a rotatable cutting tool below an upper surface of the platform, the cutting tool to remove the tab from the machined part when the machined part is slid across the platform with the tab extending into the slot and beneath the upper surface of the platform.

2. The apparatus of claim 1, wherein the platform includes a base and a guide plate selectively connectable to and detachable from the base, the guide plate to be secured to the base, the guide plate to define a portion of the upper surface of the platform along which the machined part is to slide, an edge of the guide plate to define an edge of the slot.

3. The apparatus of claim 2, wherein the guide plate is a first guide plate, the apparatus including a second guide plate selectively connectable to and detachable from the base, the first and second guide plates to be secured to the base with a gap between the first and second guide plates, a width of the gap defining a width of the slot.

4. The apparatus of claim 3, wherein the first guide plate has a first shape, and the second guide plate has a second shape, the second shape mirroring the first shape.

5. The apparatus of claim 4, including a third guide plate to replace at least one of the first guide plate or the second guide plate, the third guide plate having a third shape that is different from the first shape and different from a second shape.

6. The apparatus of claim 3, wherein at least one of the first guide plate or the second guide plate is selectively shiftable relative to the base in a direction transverse to a length of the slot to adjust the width of the slot.

7. The apparatus of claim 2, wherein the base includes a first surface and a second surface that is recessed relative to the first surface, the second surface defining a recessed region of the base, the guide plate to be within the recessed region.

8. The apparatus of claim 7, wherein a top surface of the guide plate is to be substantially flush with the first surface of the base when secured within the recessed region.

9. The apparatus of claim 1, wherein the slot extends to an end of the platform.

10. The apparatus of claim 1, wherein the slot includes an enlarged opening at a first end of the slot to facilitate insertion of the tab into the slot.

11. The apparatus of claim 1, wherein an uppermost tip of the cutting tool is to be flush with or below the upper surface of the platform.

12. The apparatus of claim 1, wherein a height of the cutting tool is adjustable relative to the upper surface of the platform.

13. The apparatus of claim 1, including a motor to cause the cutting tool to rotate on a spindle, the spindle substantially perpendicular to the upper surface of the platform.

14. The apparatus of claim 1, including a guard arm, the guard arm rotatable relative to the platform between a first position and a second position, the guard arm to extend over the cutting tool in the first position, the guard arm to be spaced apart from the cutting tool in the second position.

15. The apparatus of claim 14, including a spring to urge the guard arm toward the first position while permitting the guard arm to move to the second position in response to the machined part being slid across the platform.

16. The apparatus of claim 1, wherein the tab is a curved tab, and the slot has a shape to receive the curved tab.

17. An apparatus comprising:
a first plate including a first edge;
a second plate including a second edge;
a base to support the first and second plates with the first edge facing towards the second edge, a distance between the first and second edges defining a slot to guide a tab protruding from a machined part; and
a cutting tool to be located along the slot so as to remove the tab from the machined part as the tab is guided along the slot.

18. The apparatus of claim 17, wherein the first plate includes a first notch along the first edge, and the second plate includes a second notch along the second edge, the first notch to align with the second notch to define an opening therebetween, the cutting tool in the opening.

19. An apparatus comprising:
a cutting tool;
a motor to cause the cutting tool to spin; and
a platform including an elongate slot to guide a tab protruding from a machined part towards the cutting tool, an uppermost tip of the cutting tool to be at or below an upper surface of the platform.

20. The apparatus of claim 19, including a first guide plate to define a first side of the elongate slot, and a second guide plate to define a second side of the elongate slot, the cutting tool to be between the first and second guide plates.
